(19) United States
(12) Patent Application Publication
(10) Pub. No.: US 2022/0380723 A1
(43) Pub. Date: Dec. 1, 2022

(54) METHODS TO PRODUCE DEFINED, SPHERICAL, BIO-DEGRADABLE MACROPOROUS MICROCARRIER/HYDROGELS FOR CELLULAR AGRICULTURE

(71) Applicant: Innocent Meat GmbH, Papendorf (DE)

(72) Inventors: Patrick Nonnenmacher, Rostock (DE); Philipp Drescher, Rostock (DE)

(73) Assignee: Innocent Meat GmbH, Papendorf (DE)

(21) Appl. No.: 17/751,943

(22) Filed: May 24, 2022

Related U.S. Application Data
(60) Provisional application No. 63/290,659, filed on Dec. 17, 2021.
(60) Provisional application No. 63/192,700, filed on May 25, 2021.

(51) Int. Cl. C12N 5/00; C12N 5/077; A23L 13/00

(52) U.S. Cl. CPC C12N 5/0075; C12N 5/0659; A23L 13/00; C12N 2531/00; A23V 2002/00

(57) ABSTRACT

Biocompatible macroporous microcarriers, including microcarrier beads, microspheres, capsules, microsponges, hydrogels and other matrix forms, appropriate for use in a shaking flask or bioreactor to culture cells are described herein that can be used to create an edible structure for consumption or research investigation. Biocompatible, macroporous microcarriers can be dissolved or remain in the final product. Biocompatible macroporous microcarriers are formed by saccharides that are cross-linked via chemical induction with agitated cryo-gelation. Cross-linked macroporous, saccharide-microcarriers are coupled to adherence factors that enable cell binding. Finally, the cells are attached to the microcarrier for proliferation.

### CROSS-REFERENCE TO RELATED APPLICATIONS SECTION

This application is a U.S. Non-Provisional Patent Application that claims priority to U.S. Provisional Patent Application Ser. No. 63/290,659 filed on Dec. 17, 2021 and U.S. Provisional Patent Application Ser. No. 63/192,700 filed on May 25, 2021, the entire contents of which are hereby incorporated by reference in their entirety.

### FIELD OF THE EMBODIMENTS

The field of the invention and its embodiments relate to biocompatible (e.g., fit for human consumption or injection) microcarriers for culture and growth of cells and the formation of cultured meats, as well as methods for creating them and using them to form cultured meat products.

### BACKGROUND OF THE EMBODIMENTS

Muscle tissue engineering in vitro may provide new treatments for skeletal muscle diseases, such as muscular dystrophies or trauma, and for the generation of meat derived from livestock animal cells, for human consumption, which is referred to as “cell-based meat,” “cultured meat,” “in vitro meat,” or “cellular agriculture” (Sisma, 2019). Developing cell-based meat provides the potential to decrease resource intensity and increase environmental sustainability of meat production, compared to current industrial animal farming, which is associated with issues of greenhouse gas emission, land usage, deforestation, biodiversity, antibiotic resistance, and animal welfare (Tuomisto, 2019). (Godfray, H. C. J., Aveyard, P., Garnett, T., Hall, J. W., Key, T. J., Lorimer, J., . . . Jebb, S. A., 2018). (Cederberg, C., Persson, U. M., Neovius, K., Molander, S., & Clift, R., 2011). (Machovina, B., Feeley, K. J., & Ripple, W. J., 2015). (Mathew, A. G., Cissell, R., & Liamthong, S., 2007). The ability to grow meat in defined bioreactor conditions also allows for a decrease in the use of steroid hormones and antibiotics, while increasing the content of health-related proteins and vitamins by defined nutrient composition of cell culture media (Jeong, S. H., Kang, D., Lim, M. W., Kang, C. S., & Sung, H. J., 2013). (Ramatla, T., Ngoma, L., Adetunji, M., & Mwanza, M., 2017). Currently, the generation of muscle tissue in large quantities is not cost-efficient, since knowledge about muscle tissue engineering is mainly related to medical applications. Therefore, more basic research regarding optimization and production of muscle tissue for food products is necessary. Additionally, knowledge from interdisciplinary fields of biotechnology, pharmaceuticals, and chemical engineering can influence this development.

Furthermore, efficient culturing of adherent cells is an ongoing issue, even with a multitude of different approaches that have been described (Rowley, J., Abraham, E., Campbell, A., Brandwein, H., & Oh, S., 2012). (Cho, K. W., Kim, S. J., Kim, J., Song, S. Y., Lee, W. H., Wang, L., . . . Kim, D. H., 2019). One way to produce large quantities of such cells is to use microcarriers in bioreactor-based systems. However, removing these microcarriers out of the final meat product increases cost and usage of environment harming substances. Additionally, removing cells from their carrier system often results in significant loss of viable cell mass (Moloudi, R., Oh, S., Yang, C., Teo, K. L., Lam, A. T. L., Warkiani, M. E., & Naing, M. W., 2018). (Bodiou, V., Moutsatsou, P., & Post, M. J., 2020).

The present application remedies these problems by providing methods to produce non-animal derived products for human consumption (e.g., cultured meat) without harming the environment. More specifically, the present invention provides scalable production platforms for cultured meat by using specific adherent cells as muscle and fat progenitor cells. The present invention also provides a new type of microcarrier with beneficial characteristics, such as: (1) the microcarriers being edible and digestible so they can be incorporated into the final comestible product (e.g., no synthetic material and no toxic chemical used for their formation); (2) the microcarriers being formed of an animal-free composition to assure that the final product retains its no animal kill character (e.g., no collagen, gelatin, etc.); (3) the production of the microcarriers being scalable and at low cost; (4) the microcarriers providing a beneficial surface to volume ratio to guarantee a good multiplication factor in small volumes; and (5) the microcarriers being able to bring additional features to the final product (e.g., gustatory benefit, mouthful feeling, health benefits i.e., higher content of fibers, etc.).

### Examples of Related Art Include:

EP1789063B1 describes a non-human tissue engineered meat product and a method for producing such meat product. The meat product comprises muscle cells that are grown ex vivo. The muscle cells may be grown and attached to a support structure and may be derived from any non-human cells. The meat product may also comprise other cells such as fat cells or cartilage cells, or both, that are grown ex vivo together with muscle cells.

U.S. Pat. No. 9,332,779B2 describes dehydrated, edible, high-protein food products formed of cultured muscle cells that are combined (e.g., mixed) with a hydrogel (e.g., a plant-derived polysaccharide). These food products may be formed into a chip (e.g., snack chip), that has a protein content of greater than 50%. Further, one or more flavorants may also be included.

U.S. Pat. No. 9,752,122B2 and WO2015038988A1 describe edible microcarriers, including microcarrier beads, microspheres and microsponges, appropriate for use in a bioreactor to culture cells that may be used to form a comestible engineered meat product. For example, the edible microcarriers may include porous microcarriers that may be used to grow cells (e.g., smooth muscle cells) and may be included with the cells in the final engineered meat product, without requiring modification or removal of the cells from the microcarriers. In a particular example, the edible microcarriers may be formed of cross-linked pectin, such as pectin-thiopropionylamide (PTP), and RGD-containing polypeptide, such as thiolated cardosin A.

EP2736357B9 and ES2685638T9 describe engineered meat products formed as a plurality of at least partially fused layers, where each layer comprises at least partially fused multicellular bodies comprising non-human myocytes. The engineered meat is comestible. This reference also describes multicellular bodies comprising a plurality of non-human myocytes that are adhered and/or cohered to one another. The multicellular bodies are arranged adjacently on a nutrient-permeable support substrate and are maintained in a culture to allow the multicellular bodies to at least partially fuse to form a substantially planar layer for use in formation of engineered meat.

DE102013018242B4 describes a method for cultivating cells in adhesion culture. The method includes, at least: a) dissolving or suspending a cross-linkable, biocompatible material having adhesion points for cells in a cell culture medium; b) suspending cells in the cell culture medium, which contains the cross-linkable, biocompatible material, or in a medium that contains at least one component that is required for the cross-linking of the cross-linkable, biocompatible material; c) introducing the cell suspension into a medium in drops under conditions that initiate or permit the cross-linking of the biocompatible material, wherein either the cell suspension or the medium into which the cell suspension is introduced in drops contains the cross-linkable biocompatible material; d) forming stable, preferably porous capsules from cross-linked biocompatible material, which capsules contain incorporated adherent cells; e) proliferating the adherent cells in the capsules for a specified time period; and f) breaking up the capsule material by means of a physical or chemical stimulus and releasing the cells as a cell suspension.

WO2020243695A1 describes genetically engineered mammalian cells that endogenously express one or more phytochemicals, vitamins, or therapeutic agents and are suitable for use in a cultured meat product.

U.S. Pat. No. 7,270,829B2 describes a meat product containing in vitro produced animal cells in a three dimensional form and a method for producing the meat product.

Some similar systems exist in the art. However, their means of operation are substantially different from the present disclosure, as the other inventions fail to solve all the problems taught by the present disclosure.

### SUMMARY OF THE EMBODIMENTS

The present invention and its embodiments relate to biocompatible (e.g., fit for human consumption or injection) microcarriers for culture and growth of cells and the formation of cultured meats, as well as methods for creating them and using them to form cultured meat products. Moreover, the present invention provides biocompatible, macroporous microcarriers that are produced in a holistic, scalable, and cost-efficient method.

A first embodiment of the present invention describes a method of forming a biocompatible scaffold for use as part of an engineered meat product. The method includes numerous process steps, such as: pre-freezing a reagent solution comprising a polymer (e.g., polypeptide or a polysaccharide) and particles to form a partially frozen solution. The method further includes: stirring the partially frozen solution to ensure homogeneity, subjecting the partially frozen solution to an initiator or a cross-linker, deep-freezing the partially frozen solution to form a frozen solution, and grinding the frozen solution to form a biocompatible scaffold that comprises microbeads or a microsponge. The biocompatible scaffold may also be harvested and stored. In some examples, the biocompatible scaffold is formed from a non-animal source.

In some examples, the method further includes cross-linking the polypeptide or the polysaccharide (e.g., chitosan, pectin, or alginate) with a component to form a hydrogel. A backbone of the biocompatible scaffold is formed from cross-linking the polypeptide or the polysaccharide with a component. The component for polysaccharides comprises Ca²⁺ ions. Moreover, in some examples, the method further includes: coating the backbone of the biocompatible scaffold with a cell attachment motif. The cell attachment motif comprises an RGD-peptide in repetitions or as a single peptide, recombinant collagen, laminin, or dopamine.

Further, in some examples, the biocompatible scaffold comprises the microbeads, and where a defined size of the microbeads is between approximately 0.05 mm to approximately 5 mm. In other examples, the microbeads comprise evenly distributed pores with a size between approximately 5 μm and approximately 500 μm. In further examples, the microbeads comprise a pore volume to total volume ratio of approximately 60% to approximately 99%.

In some examples, the method includes using the biocompatible scaffold in a perfused bioreactor or a shaken flask to culture adult stem cells, embryonic stem cells, or induced pluripotent stem cells as precursors for muscle, fat tissue, or connective tissue that leads to a cultured meat product. The biocompatible scaffold remains in the cultured meat product in concentrations between approximately 0.2% and approximately 5%.

In other examples, the method further includes using the biocompatible scaffold in a perfused bioreactor or a shaken flask to culture adult stem cells, embryonic stem cells, or induced pluripotent stem cells for a therapeutic usage. In further examples, the method includes: using the biocompatible scaffold in a perfused bioreactor or a shaken flask to culture adult stem cells or embryonic stem cells as precursors for at least one of muscle tissue, fat tissue, and additional supporting cells in a co-culture system to support proliferation and later differentiation. In additional examples, the method includes using the biocompatible scaffold in a culture with muscle and fat precursor cells until the muscle and fat precursor cells are grown to confluence.

In some examples, the method additionally includes: growing cells on the biocompatible scaffold, transferring the cells on the biocompatible scaffold into differentiation inducing cell culture milieu where fat tissue and muscle tissue are built, resulting in small beads covered with the fat tissue and the muscle tissue, and interconnecting the biocompatible scaffold covered with the fat tissue and the muscle tissue during and after cultivation through use of one or more additives (e.g., transglutaminase and/or fibrinogen) to increase a meat-like texture.

A second embodiment of the present invention describes a system for macroporous microcarrier production. The system includes numerous components, such as: a micro-dispenser, a tube, a first beaker, and a second beaker. The micro-dispenser houses an alginate solution. The tube has a first end disposed opposite a second end. The first end of tube is affixed to the micro-dispenser and the second end of the tube is affixed to a component that receives pressured air. The first beaker houses a cooled liquid and configured to receive dispensed droplets from the micro-dispenser that mix with cooled liquid to form frozen drops. The second beaker houses a cooled cross-linking reagent and is configured to receive the frozen drops from the first beaker such that the frozen drops mix unthawed with the cross-linking reagent to form cross-linked drops. The cross-linked drops result into porous scaffolds having a diameter of between about 0.05 mm and about 0.5 mm at at least one of room temperature and after lyophilization.

In this system, a concentration range of the alginate solution is between about 0.1% to about 5%, a pressure between about 0.1 to about 6 bar, and a temperature is between −80° C. and −5° C. Further, the cooled liquid in the first beaker is a hydrophobic solvent, such as hexane, heptane or octane. Moreover, in some examples, the cross-linking reagent comprises CaCl₂ in ethanol or other solvents that remain in a liquid state below a temperature of 0° C. In additional examples, a concentration of the cross-linking reagent is adjustable between about 0.01% and about 5%.

A third embodiment of the present invention describes a system for macroporous microcarrier production. The system includes numerous components, such as: a micro-dispenser, a tube, a wind channel or a room comprising cooled air, and a beaker. The micro-dispenser houses an alginate solution. The tube has a first end disposed opposite a second end. The first end of the tube is affixed to the micro-dispenser and the second end of the tube is affixed to a component that receives pressured air. The wind channel or the room is configured to receive the drops from the micro-dispenser such that the cooled air creates dispensed and frozen drops. The beaker houses a cross-linking reagent and is configured to receive the dispensed and frozen drops to form cross-linked drops. The cross-linked drops form a porous scaffold at room temperature.

In this system, a concentration of the alginate solution is between about 0.1% to about 5%, a pressure between about 0.1 to about 6 bar, and a temperature is between −60° C. and −5° C. In some examples, this system is a two-phase system. In other examples, the cross-linking reagent comprises CaCl₂ in ethanol or other solvents that remain in a liquid state below a temperature of 0° C.

The porous scaffold is formed from cross-linking a polysaccharide (e.g., chitosan, pectin, or alginate) with a component. A porosity of the porous scaffold is between about 60% to about 99%. Further, the porous scaffold may be functionalized for at least one of increased adherence, increased biocompatibility, and increased cell growth by chemical modification or physical modification of the polysaccharide or the porous scaffold.

In some examples, the porous scaffold is used in a perfused bioreactor or a shaken flask to culture adult stem cells or embryonic stem cells as precursors for at least one of muscle tissue, fat tissue, and additional supporting cells in a co-culture system to support proliferation and later differentiation. In other examples, the porous scaffold is used in a culture with at least one of muscle, fat, and connective tissue precursor cells until the precursor cells are grown to confluence. In further examples, cells are grown on the porous scaffold and transferred into a differentiation inducing cell culture milieu where fat tissue and muscle tissue are built, resulting in small beads covered with the fat tissue and the muscle tissue. In additional examples, the porous scaffold is used in a perfused bioreactor or a shaken flask to culture adult stem cells, embryonic stem cells or induced pluripotent stem cells for a therapeutic usage.

The present invention relates to biocompatible, macroporous microcarriers appropriate for cell culture of adult stem cells and progenitor cells that could lead to cultured meat and cultured meat products and their production process.

The present invention described herein is free from animal-products.

The present invention provides a method to reduce the costs associated with large scale cell-cultivated meat production.

The present invention described herein is produced from plant-based or trans-genetically produced biomolecules, such as saccharides or peptides.

The biomolecules of the present invention described herein are cross-linked to form a hydrogel with evenly distributed pores.

The present invention provides hydrogels that can be functionalized by additives for improved cell proliferation and differentiation.

The present invention provides hydrogels that can be produced in defined sizes from several mL up to industrial scales.

The present invention provides hydrogels that can be produced with defined pore sizes from several nanometers to several micrometers.

The present invention provides hydrogels that can be grinded to microbeads with a defined size, retaining their evenly distributed pores, with the grinded microbeads being produced in sizes between approximately 0.05 mm to approximately 5 mm, with the evenly distributed pores having a size between approximately 5 μm and approximately 500 μm, and with the pore volume to total volume ratio being between approximately 60% to approximately 99%.

The present invention described herein will be coupled to an adherence mediating factor, such as an RGD-peptide or a trans-genetically produced collagen.

The present invention described herein can be used for cultivating different cell types that can proliferate and differentiate into skeletal muscle and fat tissue for cultured meat production.

The present invention described herein can be used for cultivating different cell types that can proliferate and differentiate into any tissue of clinical interest.

The present invention described herein includes cultured meat products that incorporate microcarriers.

### BRIEF DESCRIPTION OF THE DRAWINGS

FIG. 1 depicts a schematic diagram of an adhesion process in four phases, according to at least some embodiments disclosed herein.

FIG. 2 depicts a reaction scheme for carbodiimides, according to at least some embodiments disclosed herein.

FIG. 3 depicts a chemical structure for 1-ethyl-3-(3′-dimethylamino)carbodiimide, according to at least some embodiments disclosed herein.

FIG. 4 depicts a reaction mechanism for amide formation between carboxylic acids in alginate and amines from e.g., RGD in the presence of carbodiimide EDC in an aqueous media, according to at least some embodiments disclosed herein.

FIG. 5 depicts a chemical structure for 2-[N-morpholio]ethanesulfonic acid, according to at least some embodiments disclosed herein.

FIG. 6 depicts a schematic diagram of a first system for carrier production, according to at least some embodiments disclosed herein.

FIG. 7 depicts a schematic diagram of a second system for carrier production, according to at least some embodiments disclosed herein.

FIG. 8 depicts scanning electron microscope (SEM) images showing enlargements of a scaffold at different magnifications, according to at least some embodiments disclosed herein.

FIG. 9 depicts additional SEM images showing enlargements of a scaffold at different magnifications, according to at least some embodiments disclosed herein.

FIG. 10 depicts an image of a porous scaffold, according to at least some embodiments disclosed herein.

FIG. 11 depicts another image of a porous scaffold, according to at least some embodiments disclosed herein.

FIG. 12 depicts a further image of a porous scaffold, according to at least some embodiments disclosed herein.

FIG. 13 depicts an image of a porous scaffold, according to at least some embodiments disclosed herein.

### DESCRIPTION OF THE PREFERRED EMBODIMENTS

The preferred embodiments of the present invention will now be described with reference to the drawings. Identical elements in the various figures are identified with the same reference numerals. Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Microcarriers

Cell culture techniques have become vital to the study of animal cell structure, function and differentiation, and for the production of many important biological materials, such as vaccines, enzymes, hormones, antibodies, interferons and nucleic acids. Microcarrier culture introduces new possibilities and makes possible the practical high-yield culture of anchorage-dependent cells.

In microcarrier culture, cells grow as monolayers on the surface of small spheres or as multilayers in the pores of macroporous structures that are usually suspended in culture medium by gentle stirring. By using microcarriers in simple suspension culture, fluidized or packed bed systems, yields of up to 200 million cells per milliliter are possible.

### Adhesion to Microcarriers

The adhesion of cells to culture surfaces is fundamental to both traditional monolayer culture techniques and to microcarrier culture. Since the proliferation of anchorage-dependent cells can only occur after adhesion to a suitable culture surface, it is important to use surfaces and culture procedures that enhance all of the steps involved in adhesion (Grinnell, F., 1978).

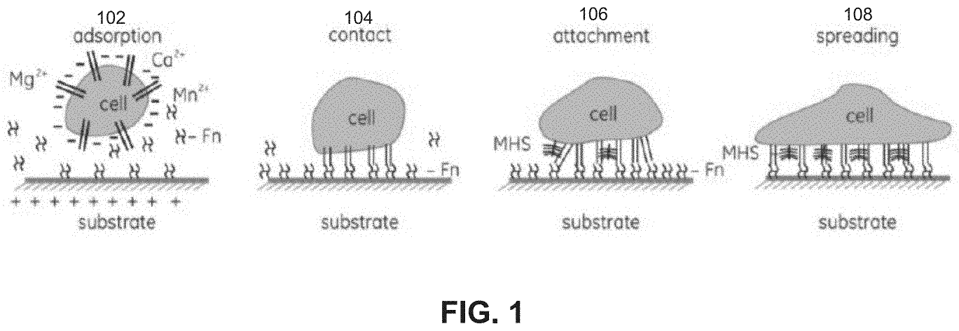

In general, attachment can be divided into four different phases, as shown in FIG. 1, with “FN” referring to fibronectin and “MHS” referring to multivalent heparan sulphate. The first phase 102 of FIG. 1 comprises a slight attachment to the surface. The second phase 104 of FIG. 1 shows flattened but still spheroidal cells, where the cell is significantly more adherent due to the increased contact area and bond density. Cells in the third phase 106 of FIG. 1 are significantly less adhesive than cells in the second phase 104 because of the reduced number of bonds. Cells in the fourth phase 108 of FIG. 1 are fully attached and extremely flat. Adhesion of cells in culture is a multi-step process and involves a) adsorption of attachment factors to the culture surface, b) contact between the cells and the surface, c) attachment of the cells to the coated surface, and finally d) spreading of the attached cells, as shown in FIG. 1 (Grinnell, 1978).

The entire process involves divalent cations and glycoproteins adsorbed to the culture surface. Under normal culture conditions, attachment proteins vitronectin and fibronectin originate from the serum supplement in the medium. MHS is synthesized by the cells. The culture surface must be hydrophilic and correctly charged before adhesion of cells can occur. All vertebrate cells possess unevenly distributed negative surface charges and can be cultured on surfaces that are either negatively or positively charged (Borysenko, J. Z. & Woods, W., 1979). (Horng, C. & McLimans, W., 1975).

Examples of suitable culture surfaces bearing charges of different polarities are glass and plastic (negatively-charged) and polylysine coated surfaces or Cytodex 1 microcarriers (positively-charged). Since cells can adhere and grow on all of these surfaces, the basic factor governing adhesion and growth of cells is the density of the charges on the culture surface rather than the polarity of the charges (Maroudas, A., 1975).

Two factors in a culture medium are essential for cells to adhere to culture surfaces: divalent cations and protein(s) in the medium or adsorbed to the culture surface (Grinnell, 1978). Many established and transformed cell types secrete only very small amounts of fibronectin and thus require a fibronectin or serum supplement in the culture medium before adhesion occurs (Grinnell, F., Hays, D. G., & Minter, D., 1977). Certain types of cells, such as diploid fibroblasts, can secrete significant quantities of fibronectin and therefore do not require an exogenous source for attachment. When initiating a culture, it is usual practice to let the culture surface come into contact with medium containing serum before cells are added to the culture. Culture medium supplemented with 10% (v/v) fetal calf serum contains approximately 2-3 μg fibronectin/mL and a large proportion of the fibronectin adsorbs to culture surfaces within a few minutes (Grinnell et al., 1977). (Ruoslahti, E. & Hayman, E. G., 1979). Serum-free media often require addition of fibronectin (1-50 μg/mL) before many cells can attach to culture surfaces.

### Materials

Materials are important because of their chemical, physical and geometrical effect on the carrier. For example, they influence toxicity, hydrophilicity, hydrophobicity, microporosity, mechanical stability, diffusion of oxygen or medium components, permeability, specific gravity, and shape (form, size, thickness, etc.). Many different natural or even synthetic biopolymers have been investigated for microcarrier formulation in regenerative medicine (Yang, Rossi, & Putnins, 2007), (M. Chen et al., 2011). In fact, alginate is often used as a bedrock biomaterial for cell transplantation due to its fast sol-gel transition in contact with divalent cations, in vivo compatibility, permeability, and dissolution (Gasperini, Mano, & Reis, 2014). Additionally, alginate has a very similar texture as the one from meat if polymerized under specific conditions. A drawback of alginate is its unsuitability for cell adhesion due to the presence of negative charges and its deficiency of integrin domains (Rowley, Madlambayan, & Mooney, 1999), (Steward, Liu, & Wagner, 2011). To overcome this inconvenience, alginate can be conjugated with a tri-amino acid sequence, arginine-glycine-asparagine (RGD), to increase its cell adhesion properties (Schmidt, Jeong, & Kong, 2011).

The most widely studied adhesive peptide in the biomaterials field is RGD. An exhaustive literature has established that RGD is highly effective at promoting the attachment of numerous cell types to a plethora of diverse materials. RGD is the principal integrin-binding domain present within ECM proteins, such as fibronectin, vitronectin, fibrinogen, osteopontin, and bone sialoprotein (Arnaout, Mahalingam, & Xiong, 2005). RGD is also present in some laminins and collagens, however RGD may be inaccessible within these molecules (depending upon conformation), and other amino acid motifs are known to serve as alternative binding modules for laminin and collagen-selective receptors (Von Der Mark, Park, Bauer, & Schmuki, 2010), (Plow, Haas, Zhang, Loftus, & Smith, 2000). The RGD sequence can bind to multiple integrin species, and synthetic RGD peptides offer several advantages for biomaterials applications. Because integrin receptors recognize RGD as a primary sequence (although conformation of the peptide can modulate affinity), the functionality of RGD is usually maintained throughout the processing and sterilization steps required for biomaterials synthesis, many of which cause protein denaturation. The use of RGD, as compared with native ECM proteins, also minimizes the risk of immune reactivity or pathogen transfer, particularly when xenograft or cadaveric protein sources are utilized.

Another benefit is that the synthesis of RGD peptides is relatively simple and inexpensive, which facilitates translation into the clinic. Finally, RGD peptides can be coupled to material surfaces in controlled densities and orientations. These advantages of straightforward synthesis, minimal cost, and tight control over ligand presentation cannot readily be achieved when using full-length native matrix proteins to functionalize material surfaces (Bellis, 2011). The present invention discusses the development of an alginate based microcarrier coated with RGD-peptides to allow cell adherence.

### Size, Shape, and Diffusion Limits and Porosity

The diameter of the different carriers varies from approximately 10 μm up to approximately 5 mm. The smaller diameters are best suited for stirred tanks, whereas the higher sedimentation rates of the larger diameters make them suitable for fluidized and packed beds. The smaller the carriers, the larger the surface in the settled bed volume because of the smaller void volume between them. The ideal size for smooth microcarriers is approximately 100 to approximately 300 μm. A very narrow size distribution is most important for good mixing in the reactor and an equal sedimentation of the beads during scale-up steps in large-scale processes. Emulsion and droplet techniques give round carriers. Macroporous carriers are on average bigger because their pores may be up to approximately 400 μm wide. A large pore size has to be balanced against the disadvantages of bigger particles, such as diffusion limits and higher shear stress on the outer surface.

The latest development in microcarrier technology is macroporous carriers that allow cells to enter. Their average pore size is between approximately 30 μm and approximately 400 μm. As the mean cell diameter of single cells in suspension is approximately 10 μm, this allows cells easy access into the carriers. Macroporous carriers are also suitable for immobilizing non-adherent cell types. In this case, the cells are forced into the matrix and entrapped. Macroporous carriers give higher cell densities and are therefore normally used in perfusion culture. The porosity of macroporous carriers is defined as the percentage volume of pores compared with the total carrier volume. It is normally between approximately 60% and approximately 99%. In spite of the large number of microcarrier designs and types, very few are still commercially available. Even fewer fulfill industrial standards for large-scale manufacturing processes.

### Production of Macroporous Microcarrier

A method for the production of large volume 3D microporous hydrogels for advanced biotechnological, medical and environmental applications is described herein (Savina, I. N., Ingavle, G. C., Cundy, A. B. & Mikhalovsky, S. V., 2016). In fact, macroporous gels can be prepared by numerous methods (Savina, Ingavle, Cundy & Mikhalovsky, 2016).

For example, a first method includes a reagent solution with monomers and particles, a frozen solution with a polymer network, and a macroporous gel (e.g., between approximately 1 mL to 10 mL). The macroporous gel can be prepared by cryogelation, involving freezing the initial gel-forming solution and carrying out polymerization or gel formation at temperatures between approximately 12° C. to approximately 18° C. below the freezing point of the solvent. Solvent (ice) crystals formed during the freezing of the solvent act as a porogen. Pores filled with water are formed after defrosting of the material.

To obtain the macroporous gel, the solvent crystals need to be formed before the gel forms. To reduce the temperature gradient and also slow down the reaction which leads to the formation of the gel, the reagent solution has to be pre-cooled in ice before adding an initiator or cross-linker, and also the initiator concentration can be reduced to slow down the polymerization itself. The macroporous gel forms after defrosting. The gel morphology depends on the cooling rate and the gel geometry.

Another method utilizes a reagent solution with monomers and particles, a partially frozen solution, a frozen solution, and a macroporous gel (e.g., between approximately 100 mL to approximately 500 mL). To effectively control the freezing of large volumes a partial freezing, or “pre-freezing” of the mixture before initiating gel formation has to be performed. It is possible to partially freeze the solution at temperatures below the solvent freezing point and with constant mixing allow the solvent to crystallize. Between 50 and 90% of the solvent can be frozen out with the gel-forming reagents remaining in the non-frozen liquid phase. Mixing the reaction solution improves the heat transfer and ice nuclei formation. This allows even freezing of large volumes of reaction solution.

However in the pre-frozen sample, most of the solvent is crystalized and the ice crystals are more evenly distributed within the sample, creating an environment close to a completely frozen block. The initiator has to be added after solution pre-freezing, thus polymerization can be delayed, and occurs in small regions of the non-frozen liquid phase, separated by ice-crystals. The heat transfer in these samples relates to the freezing out of small volumes of the reagent solution and the cooling down of the sample itself. It has been shown that the pre-freezing allows production of porous gels of large volume and near-uniform porosity along the whole volume of the sample. After formation of the frozen solution, a defrosting process step occurs to form a macroporous gel. The macroporous gel formed by this process can be sliced into small particles that can be used as suspension-microcarrier. Possible mechanisms to use include different milling techniques, such as rotor mills.

### Coating of Microcarrier

A peptide bond is formed when a carboxylic acid group of one molecule reacts with an amine group of another molecule with the release of water. In the cytosol of the cell, the formation of peptide bonds is catalyzed by enzymes. Alginate is built up of monomers that possess a carboxyl group which can form peptide bonds with the amine terminus of peptides. To manage this in vitro, carbodiimide chemistry can be used. The anchoring of RGD onto a material needs to be strong in order to induce proper cell adhesion. In theory, coupling alginates with RGD-peptides via an amide bond is a logical approach, as the peptide bond offers strong linkage between the peptide and the biomaterial.

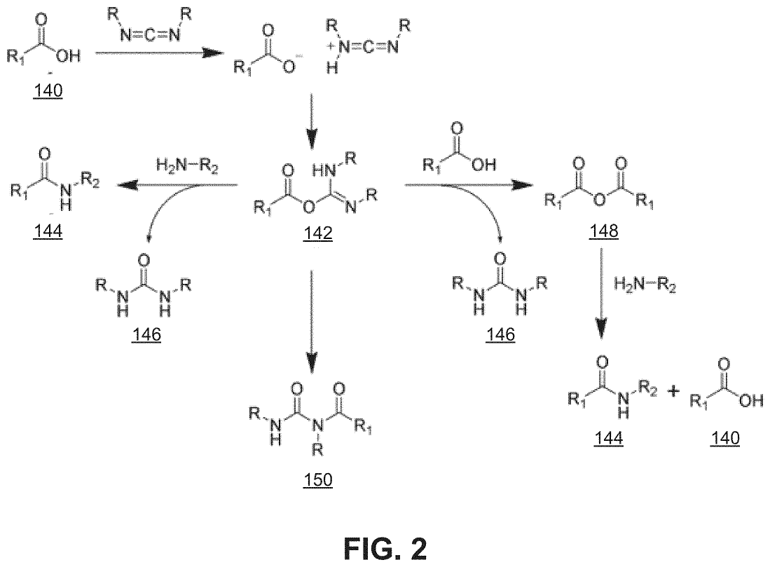

For example, carbodiimides are a collective term for unsaturated compounds with an allene structure, such as RN═C═NR. The nitrogen atoms pull the bonding electrons, resulting in a partial negative charge on the nitrogen atoms, and a corresponding positive charge on the central carbon. This creates an electrophilic carbon atom that is readily attacked by nucleophiles such as mannuronic carboxylate ions, as shown in FIG. 2.

More specifically, FIG. 2 depicts the formation of an amide using a carbodiimide. As shown in FIG. 2, an acid 140 will react with the carbodiimide to produce the key intermediate: an O-acylisourea 142, which can be viewed as a carboxylic ester with an activated leaving group. The O-acylisourea 142 will react with amines to give a desired amide 144 and a urea 146. The possible reactions of the O-acylisourea 142 produce both desired and undesired products. The O-acylisourea 142 can react with an additional carboxylic acid 140 to give an acid anhydride 148, which can react further to give the amide 144. The main undesired reaction pathway involves the rearrangement of the O-acylisourea 142 to the stable N-acylurea 150. The use of solvents with low dielectric constants such as dichloromethane or chloroform can minimize this side reaction.

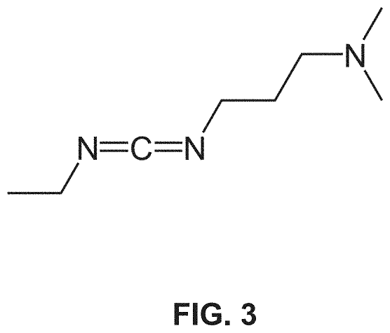

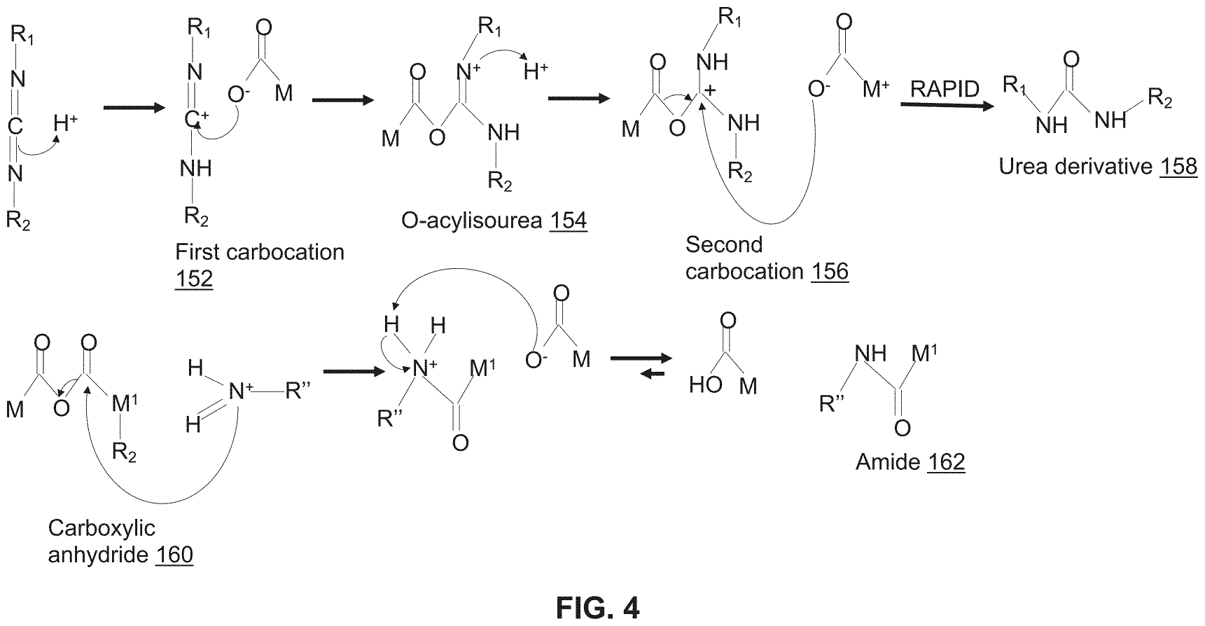

Further, N-(3-dimetylaminopropyl)-N′-ethylcarbodiimide hydrochloride (EDC) is frequently used as a carbodiimide for amide bond formation and its structure is depicted in FIG. 3. EDC reacts with mannuronic acids, as shown in FIG. 4. More specifically, FIG. 4 depicts a reaction mechanism for amide formation between carboxylic acids in alginate and amines from RGD in the presence of carbodiimide EDC in aqueous media. Protons are substrates in the carbodiimide reaction and the pH will thus influence the reaction. EDC is water soluble and very reactive, particularly in the pH-interval 3.5-4.5. At this pH, the formation of a second carbocation 156 is faster due to higher proton concentrations (as shown in FIG. 4). Mannuronic acids have a pKa of 3.38, and above this pKa, the acid groups of mannuronan are deprotonated and can act as nucleophiles as shown in FIG. 4. Below pH=3.5, the carboxylic acids of mannuronan are protonated, leading to a reduced formation of O-acylisourea 154.

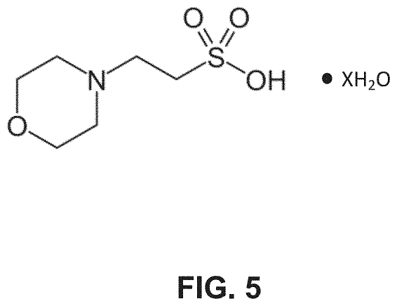

As shown in FIG. 4, a first carbocation 152 is formed by nucleophilic attack of protons. The first carbocation 152 is further attacked by nucleophilic mannuronic acids, and the O-acylisourea 154 is formed. The stoichiometry show that one proton is consumed for each O-acylisourea formed. A proton is attacked by the lone electron pair of the nitrogen atom of the O-acylisourea 154, creating the second carbocation 156. The second carbocation 156 is attacked by another nucleophilic mannuronic acid, creating a carboxylic anhydride and a urea derivate 158. A carboxylic anhydride 160 will form an amide 162 when amines are present. Consumption of protons requires a buffer to maintain the acidity of the solution. The buffer cannot have any carboxylic acids, as these will react with the O-acylisourea 154. The compound 2-[N-morpholio]ethanesulfonic acid (MES) is frequently used as a buffer in carbodiimide chemistry because it contains no carboxylic acids, and has a pKa of 6.15 at approximately 20° C. The structure of IVIES is depicted in FIG. 5. Carbodiimide chemistry allows for the use of an aqueous environment and non-hazardous reagents. Excess reagents and water-soluble urea derivates are readily removed by dialysis.

### Invention

### Scaffold

In general, the present invention provides biocompatible scaffolds (including microbeads, microsponges and hydrogels), methods for their production, and methods for their incorporation into final cultured meat products. The biocompatible microcarrier is generally formed from an animal-product-free material or materials, meaning that the material is derived from a non-animal-source. The scaffold-material is biocompatible, meaning it can be consumed by humans and other living organisms, such as pets and livestock animals. In some examples, the scaffold backbone may be built by crosslinked polypeptides or polysaccharides, such as chitosan or alginate, and may be coated or functionalized with cell-attachment-motifs, like RGD-peptides in various repetitions or as single peptide. Cells can bind to this peptide via cell surface-receptors like integrins. It should be appreciated that other peptides may be used that are not explicitly listed herein.

The biocompatible scaffold described herein includes hydrogels with evenly distributed pores, where cells can invade and grow, protected from shear stress and bead to bead collision. The size of these hydrogels can range from lab scale of several mL up to industrial scales of approximately 5000 L or more.

The hydrogel can be grinded into macroporous microbeads of a defined size between approximately 0.05 mm to approximately 5 mm, retaining their evenly distributed pores. Described pores will be evenly distributed with a size between approximately 5 μm and approximately 500 μm. The present microbeads described herein will provide a pore volume to total volume ratio of approximately 60% to approximately 99%.

Furthermore, the macroporous microcarrier can be used in a perfusion driven bioreactor in concentrations between approximately 2 g/L to approximately 30 g/L and can fill up to approximately 60% of the working volume of the bioreactor.

Additionally, the perfusion bioreactor can differ in its structure. Examples of the perfusion bioreactor include stirred tank, wave rocking, orbital shaken and gas driven bioreactors, among others not explicitly listed herein.

Further, the macroporous microcarriers described herein can be used in a lab scale shake flask for adherent cell culture investigations in concentrations between approximately 2 g/L to approximately 10 g/L.

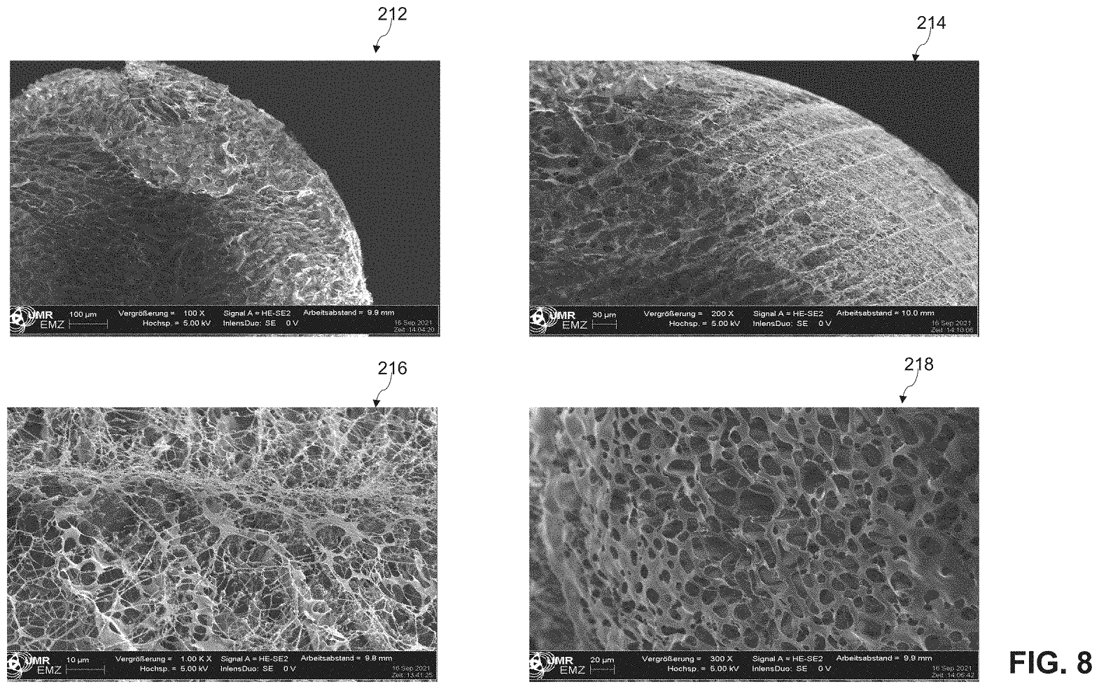

FIG. 8 depicts SEM images and enlargements of the scaffold described herein at differing magnifications. For example, FIG. 8 includes a first image 212 of the scaffold at a magnification of 100×, a second image 214 of the scaffold at a magnification of 200×, a third image 216 of the scaffold at a magnification of 1.00K×, and a fourth image 218 of the scaffold at a magnification of 300×.

### Method for Production of Biocompatible Macroporous Microcarriers

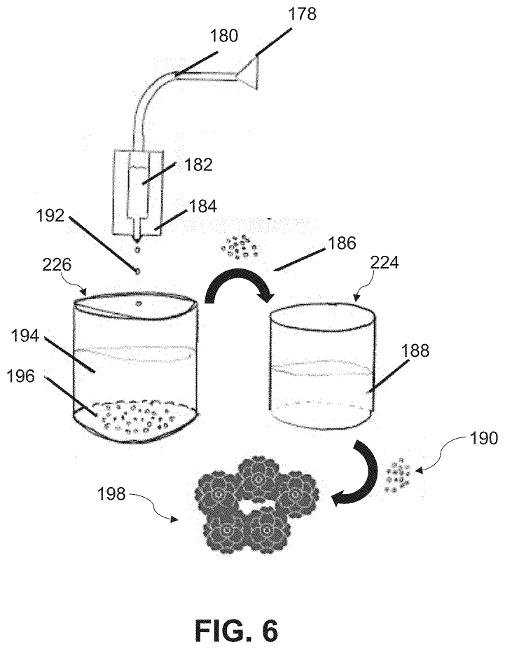

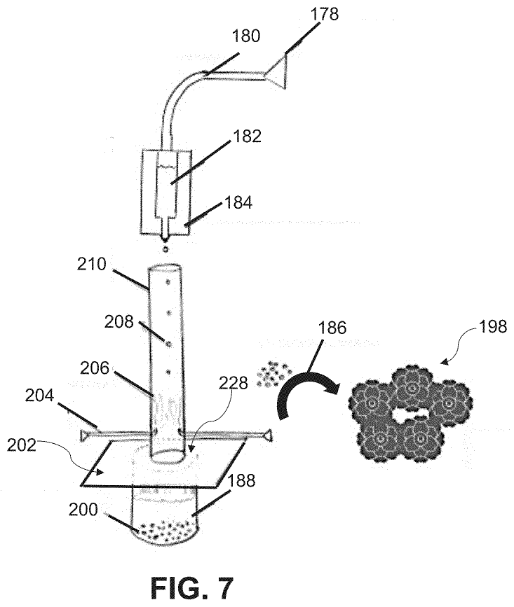

Biocompatible hydrogels with evenly, defined pores can be produced by creating evenly sized small drops that will exhibit the same cryogelation kinetics, and therefore evading the issue with inhomogeneous crystal formation, as described in FIG. 6 and FIG. 7 herein. Biocompatible materials can be presented by polypeptides or polysaccharides, such as chitosan and alginate. In an example, an alginate could be crosslinked via Ca²⁺ Ions. The corresponding anion is mediating the cross-linking velocity and thus the final texture of the cross-linked scaffold.

Macroporous gels can be prepared by cryogelation, involving freezing the initial gel-forming solution and carrying out polymerization or gel formation at temperatures between approximately 10° C. to approximately 60° C. degrees below the freezing point of the solvent.

Solvent (ice) crystals formed during the freezing of the solvent act as a porogen. Pores filled with water are formed after defrosting of the material. To obtain a macroporous gel, the solvent crystals need to be formed before the gel forms. The gel morphology depends on the cooling rate and time and the gel geometry. The macroporous gel forms after defrosting.

### Application

Present biocompatible, macroporous microcarriers could be used in perfused bioreactor or shaken flasks to culture adult stem cells or embryonic stem cells as precursors for muscle and fat tissue that could lead to cultured meat products.

Present biocompatible, macroporous microcarriers could be used in perfused bioreactor or shaken flasks to culture adult stem cells or embryonic stem cells or other cell types for therapeutic usage and possible injection into patients.

Present biocompatible, macroporous microcarriers could be used in perfused bioreactor or shaken flasks to culture adult stem cells or embryonic stem cells as precursors for muscle and fat tissue and additional supporting cells in co-culture system to support proliferation and later differentiation.

Present biocompatible, macroporous microcarriers may retentate in the final product of cultured meat in concentrations between approximately 0.2% to approximately 5%.

Present biocompatible, macroporous microcarriers may be in culture with muscle and fat precursor cells until the cells are grown to confluence, though this is not necessary. Thereby, cell transfer from bead to bead is possible and wanted.

Cells grown on present biocompatible, macroporous microcarriers may be transferred on the microcarrier into differentiation inducing cell culture milieu where fat tissue and muscle tissue will be built, resulting in small beads covered with associated tissue.

Tissue covered and filled biocompatible, macroporous microcarriers can be interconnected during and after cultivation by additives such as transglutaminase and fibrinogen to form more meat like textures.

Tissue covered and filled biocompatible, macroporous non-interconnected or connected microcarriers can be harvested and filled into bags for storage and sending. Single use bags inside of the bioreactor could serve as packaging unit after releasing excess of culture media and washing of cells.

### Systems for Carrier Production

A process method described herein includes numerous process steps, such as: preparing an alginate solution; dispensing droplets of the alginate solution; freezing and collecting the droplets in a cooled liquid; cross-linking of the scaffolds; filtering the droplets and temperature storing them in a freezer; freeze drying the droplets into scaffolds; sterilizing; acclimatizing in a DMEM media; and cell seeding.

FIG. 6 depicts a schematic diagram of a first system for carrier production. The system of FIG. 6 includes at least a micro-dispenser 184 housing an alginate solution 182. The system also includes a tube 180 having a first end disposed opposite a second end. The first end of the tube 180 is affixed to the micro-dispenser 184 and the second end of the tube 180 is affixed to a component that receives pressurized air 178. The system also includes a first beaker 226 and a second beaker 224. The first beaker 226 includes an undercooled liquid 194. The undercooled liquid 194 is configured to mix with droplets 192 dispensed from the micro-dispenser 184 to form frozen droplets 196, where the frozen droplets are transferred, at a process step 186, to the second beaker 224. The second beaker 224 includes a cross-linking agent 188, such that the cross-linked droplets are transferred, at a process step 190, to a porous scaffold 198 at room temperature.

In examples, the alginate solution ranges from about 0.1% to about 5% and the pressure ranges from about 0.1 to about 6 bar. Moreover, the temperature ranges from about −80° C. to about −10° C. Furthermore, the undercooled liquid 194 may comprise heptane, pentane, and/or hexane and the cross-linking agent 188 may comprise CaCl₂ in ethanol.

FIG. 7 depicts a schematic diagram of a second system for carrier production. Similar to the first system of FIG. 6, the second system of FIG. 7 includes at least the micro-dispenser 184 housing the alginate solution 182. The system of FIG. 7 also includes the tube 180 having a first end disposed opposite a second end. The first end of the tube 180 is affixed to the micro-dispenser 184 and the second end of the tube 180 is affixed to a component that receives the pressurized air 178. The system of FIG. 7 also includes a wind channel 210, where droplets dispensed from the micro-dispenser 184 contact cooled air 206 to form freezing droplets 208. The drops also contact pressured and cooled air 204 prior to passing a plate 202 and entering a beaker 228. It should be appreciated that the wind channel 210 may be cooled such that it provides an alternative to the undercooled liquid 194 of FIG. 6. The beaker 228 houses a cross-linking agent 188, which results in cross-linked droplets 200. The cross-linked drops or droplets 200 are transferred, at a process step 186, to porous scaffolds 198 at room temperature.

It should be appreciated, that in some examples, the alginate solution ranges from about 0.1% to about 2% and the pressure ranges from about 0.1 to about 6 bar. Moreover, the temperature ranges from about −60° C. to about −1° C. Furthermore, the cross-linking agent 188 may comprise CaCl₂ in ethanol.

With regards to FIG. 6 and FIG. 7, in some examples, the frozen drops or droplets may be created with a cooled solvent (such as n-heptane or pentane). In some examples, the alginate concentrations may vary between about 0.25% to about 2%. Further, in other embodiments, the temperature of the solvent and/or the alginate solution may vary.

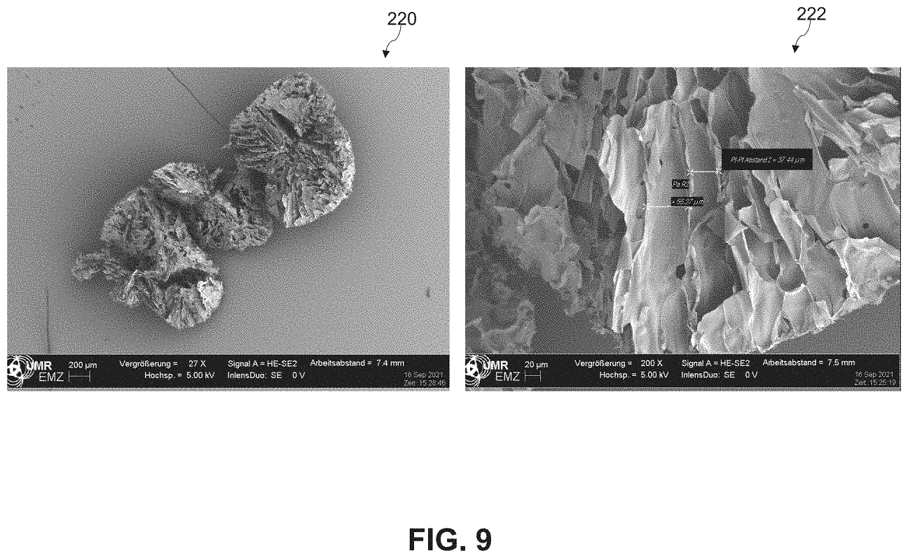

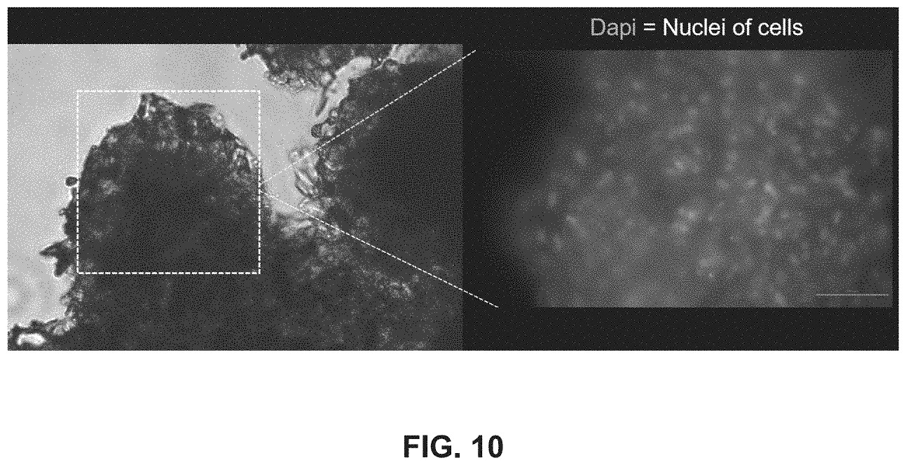

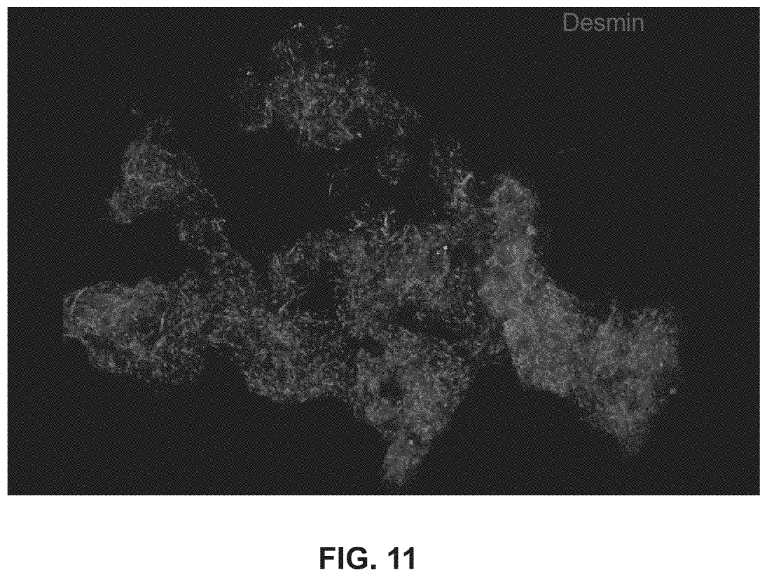

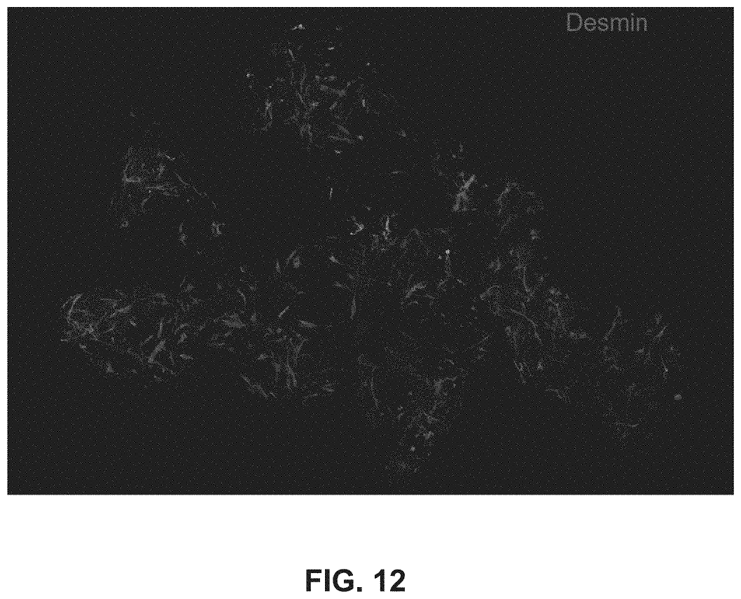

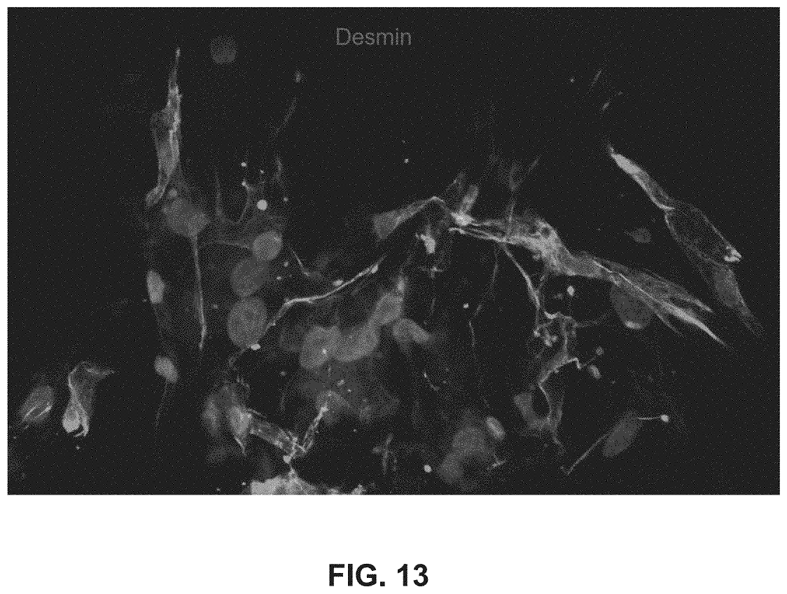

FIG. 9 depicts SEM images and enlargements of the scaffold described herein at differing magnifications. Moreover, FIG. 9, depicts a first image 220 of the scaffold at a magnification of 27× and a second image 222 of the scaffold at a magnification of 200×. FIG. 9 showcases the open porous structure of the scaffolds, with larger radial channels of about ˜50 μm. Further, FIG. 9 depicts a fine mesh of alginate filaments on the surface and some minor inhomogeneities throughout the drops or droplets. FIG. 10-FIG. 13 also depict images of the porous scaffold described herein.

In additional examples, Brunauer—Emmett—Teller (BET) measurements and porosity measurements may be taken for further analysis. BET theory aims to explain the physical adsorption of gas molecules on a solid surface and serves as the basis for an important analysis technique for the measurement of the specific surface area of materials. The observations are very often referred to as physical adsorption or physisorption.

### EXAMPLES

### Example 1

Satellite cells are cultured in an approximately 1000 L scale on a present biocompatible macroporous microcarrier with a concentration of approximately 20 g/L. A commercially available macroporous microcarrier achieved maximum cell densities of approximately 2×10⁸ cells/mL. Calculating with one fourth of this density leads to cell masses of approximately 0.5×10¹⁴ cells with approximately 20 kg of microcarrier in the final product. Given the information that approximately 1-2×10⁸ cells results in approximately 1 g of meat, the final product weight results in approximately 0.5×10⁶ g or 500 kg pure meat with approximately 20 kg microcarrier. The final concentration of microcarriers is thus approximately 5%.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

Arnaout, M. A., Mahalingam, B., & Xiong, J. P. (2005). Integrin structure, allostery, and bidirectional signaling. Annu. Rev. Cell. Dev. Biol. DOI: 10.1146/annurev.cellbio.21.090704.151217.

Bellis, S. L. (2011). Advantages of RGD peptides for directing cell association with biomaterials. Biomaterials. DOI: 10.1016/j .biomaterials.2011.02.029.

Bodiou, V., Moutsatsou, P., & Post, M. J. (2020). Microcarriers for upscaling cultured meat production. Frontiers in Nutrition. DOI: 10.3389/fnut.2020.00010.

Borysenko, J.Z. & Woods, W. (1979). Density, distribution and mobility of surface anions on a normal/transformed cell pair. Exp. Cell Res. DOI: 10.1016/0014-4827(79)90146-0.

Cederberg, C., Persson, U. M., Neovius, K., Molander, S., & Clift, R. (2011). Including carbon emissions from deforestation in the carbon footprint of Brazilian beef. Environmental Science and Technology. DOI: 10.1021/es103240z.

Chen, I., Hill, J. K., Ohlemueller, R., Roy, D. B., & Thomas, C. D. (2011). Rapid Range Shifts of Species Associated with High Levels of Climate Warming. Science. DOI: 10.1126/science.1206432.

Cho, K. W., Kim, S. J., Kim, J., Song, S. Y., Lee, W. H., Wang, L., . . . Kim, D. H. (2019). Large scale and integrated platform for digital mass culture of anchorage dependent cells. Nature Communications. DOI: 10.1038/s41467-019-12777-3.

Gasperini, L., Mano, J. F., & Resis, R. L. (2014). Natural polymers for the microencapsulation of cells. J. R. Soc. Interface. DOI: 10.1098/rsif.2014.0817.

Grinnell, F., Hays, D. G., & Minter, D. (1977). Cell adhesion and spreading factor: Partial purification and properties. Experimental Cell Research. DOI: 10.1016/0014-4827(77)90284-1.

Grinnell, F. (1978). Cellular Adhesiveness and Extracellular Substrata. International Review of Cytology. DOI: 10.1016/50074-7696(08)62241-X.

Godfray, H. C. J., Aveyard, P., Garnett, T., Hall, J. W., Key, T. J., Lorimer, J., . . . Jebb, S. A. (2018). Meat consumption, health, and the environment. Science (New York, N.Y.). DOI: 10.1126/science.aam5324.

Horng, C. & McLimans, W. (1975). Primary suspension culture of calf anterior pituitary cells on a microcarrier surface. Biotechnology & Bioengineering. DOI: 10.1002/bit.260170508.

Jeong, S. H., Kang, D., Lim, M. W., Kang, C. S., & Sung, H. J. (2013). Risk assessment of growth hormones and antimicrobial residues in meat. Toxicological Research. DOI: 10.5487/TR.2010.26.4.301.

Machovina, B., Feeley, K. J., & Ripple, W. J. (2015). Biodiversity conservation: The key is reducing meat consumption. Science of the Total Environment. DOI: 10.1016/j.scitotenv.2015.07.022.

Maroudas, A. (1975). Biophysical chemistry of cartilaginous tissues with special reference to solute and fluid transport. Biorheology. DOI: 10.3233/bir-1975-123-416.

Mathew, A. G., Cissell, R., & Liamthong, S. (2007), Antibiotic resistance in bacteria associated with food animals: A United States perspective of livestock production. Foodborne Pathogens and Disease. DOI: 10.1089/fpd.2006.0066.

Moloudi, R., Oh, S., Yang, C., Teo, K. L., Lam, A. T. L., Warkiani, M. E., & Naing, M. W. (2018). Inertial-Based Filtration Method for Removal of Microcarriers from Mesenchymal Stem Cell Suspensions. Scientific Reports. DOI: 10.1038/s41598-018-31019-y.

Plow, E. F., Haas, T. A., Zhang, L., Loftus, J., & Smith, J. W. (2000). Ligand binding to integrins. J. Biol. Chem. DOI: 10.1074/jbc.R000003200.

Ramatla, T., Ngoma, L., Adetunji, M., & Mwanza, M. (2017). Evaluation of antibiotic residues in raw meat using different analytical methods. Antibiotics. DOI: 10.3390/antibiotics6040034.

Rowley, J. A., Madlambayan, G., & Mooney, D. J. (1999). Alginate hydrogels as synthetic extracellular matrix materials. Biomaterials. DOI: 10.1016/s0142-9612(98)00107-0.

Rowley, J., Abraham, E., Campbell, A., Brandwein, H., & Oh, S. (2012). Meeting lot-size challenges of manufacturing adherent cells for therapy. BioProcess International.

Ruoslahti, E. & Hayman, E. G. (1979). Two active sites with different characteristics in fibronectin. FEBS Lett. DOI: 10.1016/0014-5793(79)80088-5.

Savina, I. N., Ingavle, G. C., Cundy, A. B. & Mikhalovsky, S. V. (2016). A simple method for the production of large volume 3D microporous hydrogels for advanced biotechnological, medical and environmental applications. Nature. Scientific Reports. DOI: 10.1038/srep21154.

Schmidt, J. J., Jeong, J. H., Cha, C., & Kong, H. (2011). Controlling the dependency between hydrogel rigidity and permeability with the inflexibility of a polymer cross-linker. Materials Engineering and Sciences Division—Core Programming Topic at the 2011 AIChE Annual Meeting (Materials Engineering and Sciences Division—Core Programming Topic at the 2011 AIChE Annual Meeting; Vol. 1).

Simsa, R., Yuen, J., Stout, A., Rubio, N., Fogelstrand, P., & Kaplan, D. L. (2019). Extracellular heme proteins influence bovine myosatellite cell proliferation and the color of cell-based meat. Foods. DOI: 10.3390/foods8100521.

Steward, A. J., Liu, Y., & Wagner, D. R. (2011). Engineering cell attachments to scaffolds in cartilage tissue engineering. Biomaterials for Regenerative Medicine. DOI: 0.1007/s11837-011-0062-x.

Tuomisto, H. L. (2019). The eco-friendly burger. EMBO Reports. DOI: 10.15252/embr.201847395.

Von Der Mark, K., Park, J., Bauer, S., & Schmuki, P. (2010). Nanoscale engineering of biomimetic surfaces: cues from the extracellular matrix. Cell Tissue Res. DOI: 10.1007/s00441-009-0896-5.

Yang, Y., Rossi, F. M. V., & Putnins, E. E. (2007). Ex vivo expansion of rat bone marrow mesenchymal stromal cells on microcarrier beads in spin culture. Biomaterials. DOI: 10.1016/j.biomaterials.2007.03.015.

What is claimed is:

1. A method of forming a biocompatible scaffold for use as part of an engineered meat product, the method comprising:
pre-freezing a reagent solution comprising a polymer and particles to form a partially frozen solution;
stirring the partially frozen solution to ensure homogeneity;
subjecting the partially frozen solution to an initiator or a cross-linker;
deep-freezing the partially frozen solution to form a frozen solution; and
grinding the frozen solution to form a biocompatible scaffold that comprises microbeads or a microsponge.

2. The method of claim 1, wherein the polymer comprises a polypeptide or a polysaccharide.

3. The method of claim 2, further comprising:
cross-linking the polypeptide or the polysaccharide with a component to form a hydrogel.

4. The method of claim 2, wherein the polysaccharide is selected from the group consisting of: chitosan, pectin, and alginate.

5. The method of claim 2, wherein a backbone of the biocompatible scaffold is formed from cross-linking the polypeptide or the polysaccharide with a component, and wherein the component for polysaccharides comprises Ca²⁺ ions.

6. The method of claim 5, further comprising:
coating or covalent coupling the backbone of the biocompatible scaffold with a cell attachment motif, wherein the cell attachment motif comprises an RGD-peptide in repetitions or as a single peptide, recombinant collagen, laminin, tyramine or dopamine.

7. The method of claim 6, wherein the biocompatible scaffold comprises the microbeads, and wherein a defined size of the microbeads is between approximately 0.05 mm to approximately 5 mm.

8. The method of claim 6, wherein the biocompatible scaffold comprises the microbeads, and wherein the microbeads comprise evenly distributed pores with a size between approximately 5 μm and approximately 500 μm.

9. The method of claim 6, wherein the biocompatible scaffold comprises the microbeads, and wherein the microbeads comprise a pore volume to total volume ratio of approximately 60% to approximately 99%.

10. The method of claim 6, further comprising:
using the biocompatible scaffold in a perfused bioreactor or a shaken flask to culture adult stem cells, embryonic stem cells, or induced pluripotent stem cells as precursors for muscle, fat tissue, or connective tissue that leads to a cultured meat product.

11. The method of claim 10, wherein the biocompatible scaffold remains in the cultured meat product in concentrations between approximately 0.2% and approximately 5%.

12. The method of claim 6, further comprising:
using the biocompatible scaffold in a perfused bioreactor or a shaken flask to culture adult stem cells, embryonic stem cells, or induced pluripotent stem cells for a therapeutic usage.

13. The method of claim 6, further comprising:
using the biocompatible scaffold in a perfused bioreactor or a shaken flask to culture adult stem cells or embryonic stem cells as precursors for at least one of muscle tissue, fat tissue, and additional supporting cells in a co-culture system to support proliferation and later differentiation.

14. The method of claim 6, further comprising:
using the biocompatible scaffold in a culture with muscle and fat precursor cells until the muscle and fat precursor cells are grown to confluence.

15. The method of claim 6, further comprising:
growing cells on the biocompatible scaffold; and
transferring the cells on the biocompatible scaffold into differentiation inducing cell culture milieu where fat tissue and muscle tissue are built, resulting in small beads covered with the fat tissue and the muscle tissue.

16. The method of claim 15, further comprising:
interconnecting the biocompatible scaffold covered with the fat tissue and the muscle tissue during and after cultivation through use of one or more additives to increase a meat-like texture.

17. The method of claim 16, wherein the one or more additives are selected from the group consisting of: transglutaminase and fibrinogen.

18. The method of claim 6, further comprising:
harvesting the biocompatible scaffold; and
storing the biocompatible scaffold.

19. The method of claim 1, wherein the biocompatible scaffold is formed from a non-animal source.

20. A system for macroporous microcarrier production comprising:
a micro-dispenser housing an alginate solution;
a tube having a first end disposed opposite a second end, the first end of tube being affixed to the micro-dispenser and the second end of the tube being affixed to a component that receives pressured air;
a first beaker housing a cooled liquid and configured to receive dispensed droplets from the micro-dispenser that mix with cooled liquid to form frozen drops; and
a second beaker housing a cooled cross-linking reagent and configured to receive the frozen drops from the first beaker such that the frozen drops mix unthawed with the cross-linking reagent to form cross-linked drops, wherein the cross-linked drops result into porous scaffolds having a diameter of between about 0.05 mm and about 0.5 mm at at least one of room temperature and after lyophilization.

21. The system of claim 20, wherein a concentration range of the alginate solution is between about 0.1% to about 5%, wherein a pressure between about 0.1 to about 6 bar, and wherein a temperature is between −80° C. and −5° C.

22. The system of claim 20, wherein the cooled liquid in the first beaker is a hydrophobic solvent, and wherein the hydrophobic solvent is selected from the group consisting of hexane, heptane and octane.

23. The system of claim 20 wherein the cross-linking reagent comprises CaCl₂ in ethanol or other solvents that remain in a liquid state below a temperature of 0° C.

24. The system of claim 20, wherein a concentration of the cross-linking reagent is adjustable between about 0.01% and about 5%.

25. A system for macroporous microcarrier production, the system comprising:
a micro-dispenser housing an alginate solution;
a tube having a first end disposed opposite a second end, the first end of the tube being affixed to the micro-dispenser and the second end of the tube being affixed to a component that receives pressured air;
a wind channel or a room comprising cooled air, such that the wind channel or the room is configured to receive the drops from the micro-dispenser and the cooled air creates dispensed and frozen drops; and
a beaker housing a cross-linking reagent and configured to receive the dispensed and frozen drops to form cross-linked drops, wherein the cross-linked drops form a porous scaffold at room temperature.

26. The system of claim 25, wherein a concentration of the alginate solution is between about 0.1% to about 5%, wherein a pressure between about 0.1 to about 6 bar, and wherein a temperature is between −60° C. and −5° C.

27. The system of claim 25, wherein the system is a two-phase system.

28. The system of claim 25, wherein the cross-linking reagent comprises CaCl₂ in ethanol or other solvents that remain in a liquid state below a temperature of 0° C.

29. The system of claim 25, wherein the porous scaffold is formed from cross-linking a polysaccharide with a component.

30. The system of claim 29, wherein the polysaccharide is selected from the group consisting of chitosan, pectin, and alginate.

31. The system of claim 29, wherein the porosity of the porous scaffold is between about 60% to about 99%.

32. The system of claim 29, wherein the porous scaffold is functionalized for at least one of increased adherence, increased biocompatibility, and increased cell growth by chemical modification or physical modification of the polysaccharide or the porous scaffold.

33. The system of claim 25, wherein the porous scaffold is used in a perfused bioreactor or a shaken flask to culture adult stem cells or embryonic stem cells as precursors for at least one of muscle tissue, fat tissue, and additional supporting cells in a co-culture system to support proliferation and later differentiation.

34. The system of claim 25, wherein the porous scaffold is used in a culture with at least one of muscle, fat, and connective tissue precursor cells until the precursor cells are grown to confluence.

35. The system of claim 25, wherein cells are grown on the porous scaffold and transferred into a differentiation inducing cell culture milieu where fat tissue and muscle tissue are built, resulting in small beads covered with the fat tissue and the muscle tissue.

36. The system of claim 25, wherein the porous scaffold is used in a perfused bioreactor or a shaken flask to culture adult stem cells, embryonic stem cells or induced pluripotent stem cells for a therapeutic usage.
